SEQUENCE LISTING (1 sequence)

SEQ ID NO: 1 - protein, 116 aa, Artificial sequence
  Synthetic construct; ZZ tag capable of binding Fc region of IgG
VDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPK

(19) United States
(12) Patent Application Publication
(10) Pub. No.: US 2010/0292445 A1
(43) Pub. Date: Nov. 18, 2010

(54) Method for Efficient Purification of Bionanocapsule

(76) Inventors: Shunichi Kuroda, Osaka (JP); Masumi Maekawa, Hyogo (JP); Mana Nagita, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/223,864

(22) PCT Filed: Feb. 9, 2007
(86) PCT No.: PCT/JP2007/052414

§ 371 Date: Aug. 12, 2008

(30) Foreign Application Priority Data
Feb. 13, 2006 (JP) 2006-035666

(51) Int. Cl. C07K 1/22; C07K 14/005

(52) U.S. Cl. 530/413; 530/350

(57) ABSTRACT

The present invention provides a method for purifying a heat-resistant bio-nanocapsule composed mainly of a protein produced by using a eukaryotic cell. The method comprises heat-treating a lysate of the eukaryotic cell to remove contaminant proteins, and then subjecting the resulting product to chromatography.

### TECHNICAL FIELD

The present invention relates to efficient purification and preservation methods for bio-nanocapsules.

### BACKGROUND ART

It is known that particles derived from virus proteins, such as hepatitis B virus particles, can be used as bio-nanocapsules that are usable as carriers in vivo.

For example, the inventors of the present invention have reported that by expressing a hepatitis B virus surface antigen (HBsAg) L protein in recombinant yeast cells, ellipsoidal bio-nanocapsules with a large number of L proteins embedded in yeast-derived lipid bilayer membranes can be produced (see Non-Patent Document 1). Because such particles contain no HBV genome, they do not function as viruses and are highly safe for the human body.

However, such bio-nanocapsules are produced by using non-human eukaryotic cells. Therefore, to obtain bio-nanocapsules from the cell lysate, it is necessary to remove many contaminant proteins.

For example, Patent Document 1 discloses expressing a hepatitis B virus surface antigen (HBsAg) protein in recombinant yeast cells to produce HBsAg particles (bio-nanocapsules), and then purifying the biocapsules by a combination of purification methods, such as cell disruption, cesium chloride density gradient centrifugation, dialysis, and sucrose density gradient centrifugation.

However, when purification is carried out by a density gradient centrifugation, mass production is difficult in terms of time and cost, and a more efficient method of purifying bio-nanocapsules has been desired.
Patent Document 1: Japanese Unexamined Patent Publication No. 2004-2313.
Non-Patent Document 1: J. Biol. Chem., Vol. 267, No. 3, 1953-1961, 1992.

### DISCLOSURE OF THE INVENTION

### Problem to be Solved by the Invention

An object of the present invention is to purify and preserve bio-nanocapsules using a simple, industrially applicable method.

### Means for Solving the Problem

As a result of extensive research in view of the above problem, the present inventor found that bio-nanocapsules produced used eukaryotic cells can be easily purified through thermal denaturation and precipitation of contaminant particles by heat treatment.

The inventors further found that, after heat treatment, bio-nanocapsules are further purified by chromatography methods such as Sulfate-Cellulofine chromatography and gel filtration chromatography to remove yeast- or other eukaryotic cell-derived contaminant proteins.

The present invention provides the following methods for purifying heat-resistant bio-nanocapsules, and methods for preserving the same.
1. A method for purifying a heat-resistant bio-nanocapsule composed mainly of a protein produced used a eukaryotic cell, the method comprising heat-treating a eukaryotic cell lysate to remove contaminant proteins, and then subjecting the resulting product to chromatography.
2. A method according to item 1, wherein the chromatography comprises affinity chromatography and/or gel filtration chromatography.
3. A method according to item 2, wherein the affinity chromatography is Sulfate-Cellulofine chromatography using a sulfate group-containing medium.
4. A method according to item 2, wherein the affinity chromatography is antibody affinity chromatography using a medium having an anti-HBsAg antibody bound thereto, the anti-HBsAg antibody being produced by using HBsAg particles as an antigen.
5. A method according to item 2, wherein the affinity chromatography is chromatography using hydroxyapatite as a medium.
6. A method according to any one of items 1 to 5, wherein the heat-resistant bio-nanocapsule is a capsule composed mainly of a hepatitis B virus surface antigen (HBsAg) protein.
7. A method according to any one of items 1 to 6, wherein the heat treatment is performed at 65° C. to 90° C.
8. A method for preserving a heat-resistant bio-nanocapsule, comprised of freezing the bio-nanocapsule, followed by drying under vacuum.
9. A method according to item 8, wherein the bio-nanocapsule is quickly frozen with a stabilizing agent.
10. A method according to item 8 or 9, wherein the quick-freezing temperature is not higher than −80° C.

### EFFECT OF THE INVENTION

The heat-resistant bio-nanocapsule composed mainly of a protein produced used a eukaryotic cell contains contaminant particles, and eukaryotic cell-derived contaminant proteins.

Regarding these contaminants, most of the particle impurities and a considerable amount of contaminant proteins can be removed by heat treatment, whereby greatly enhanced bio-nanocapsule purity can be obtained.

Further, the bio-nanocapsules thus heat treated can be highly purified by chromatographic methods, such as affinity chromatography and/or gel filtration chromatography using a cellulofine sulfate-containing medium, a hydroxyapatite-containing medium or an anti-HBsAg antibody-bound medium, without using cesium chloride or sucrose density gradient ultracentrifugation, thereby establishing a method of purifying bio-nanocapsules on a large industrial scale.

Further, by quickly freezing and/or lyophilizing the heat-resistant bio-nanocapsule in the presence of a saccharide, the preservation stability of the bio-nanocapsule can be greatly enhanced.

### BRIEF DESCRIPTION OF THE DRAWINGS

FIG. 1 shows an outline of the purification method according to the present invention, and a conventional purification method.

FIG. 2 shows the results of SDS-PAGE obtained before and after the heat treatment. Lane M: molecular weight marker.

FIG. 3 shows the results of Sulfate-Cellulofine chromatography. Lane M: a molecular weight marker.

FIG. 4 shows the results of gel filtration chromatography. *1: Sulfate-Cellulofine-Recovered fractions; Lane M: molecular weight marker.

FIG. 5 shows the results of SDS-PAGE obtained throughout the process. Lane M: molecular weight marker.

FIG. 6 shows the results of preservation stability of the particles. Lane M: Molecular weight marker.

FIG. 7 shows the results of preservation stability of a lyophilizate stored at 4° C. In FIG. 7, the non-saccharide control is a bio-nanocapsule purified in the absence of sugar by the method of the present invention.

### BEST MODE FOR CARRYING OUT THE INVENTION

Examples of eukaryotic cells that can be used to produce the protein include yeasts, insect cells (e.g., Sf9, Sf21, and High Five cells), mammalian cells (e.g., CHO, HEK293, and COS cells), and the like. Yeasts are preferably used. Examples of yeasts include Japanese sake yeast, shochu yeast, brewer's yeast, wine yeast, baker's yeast, and other yeasts of the genera Saccharomyces, Kluyveromyces, Pichia, and the like. Brewer's yeast and baker's yeast are particularly preferable.

When an HBsAg protein or like protein capable of forming heat-resistant bio-nanocapsules is expressed in a eukaryotic cell, the protein is expressed and accumulated as a membrane protein on the endoplasmic reticulum membrane, and bio-nanocapsules are released as nanoparticles. More specifically, plasmids, which are genetic constructs capable of expressing the protein, are introduced into eukaryotic cells to transform the cells, and the obtained eukaryotic transformant cells are cultured in a medium to produce heat-resistant bio-nanocapsules. Methods for producing bio-nanocapsules are described in WO01/64930, WO03/082330, and WO03/082344. A method for preparing HBsAg is described in Vaccine, 2001 Apr. 30; 19(23-24): 3154-63, Physicochemical and immunological characterization of hepatitis B virus envelope particles exclusively consisting of the entire L (pre-S1+pre-S2+S) protein, Yamada T., Iwabuki H., Kanno T., Tanaka H., Kawai T., Fukuda H., Kondo A., Seno M., Tanizawa K., Kuroda S. The contents of these references are incorporated herein by reference. Bio-nanocapsules can be obtained by disrupting cultured eukaryotic cells (transformants). However, the obtained bio-nanocapsules contain eukaryotic cell-derived contaminant proteins, and particle impurities that fail to function as media for introducing materials into cells.

The heat-resistant bio-nanocapsule is stable when heated at 50° C. or more, preferably 55° C. or more, and particularly 60° C. or more. The heat-resistant bio-nanocapsule may be any nanocapsule comprising a heat-resistant protein that can be heat-treated. For example, a nanocapsule composed mainly of hepatitis B virus surface antigen (HBsAg) protein can be used. Examples of nanocapsules composed mainly of HBsAg include nanocapsules comprising 70 to 85 wt. % of an HBsAg protein and 10 to 15 wt. % of a lipid, and those further comprising other proteins, such as hepatitis B virus core antigen (HBcAg) protein. The HBsAg protein includes natural proteins such as S protein, M protein, and L protein, and various mutants thereof.

The S protein (226 amino acid residues), which is included in HBsAg protein and is the component of S particle, has a particle-forming ability. The M protein (a constituent protein of M particle) is obtained by adding Pre-S2 composed of 55 amino acid residues to the S particle. The L protein (a constituent protein of L particle) is obtained by adding Pre-S1 composed of 108 amino acid residues (subtype y) or 119 amino acid residues (subtype d) to the M protein.

In this specification, the amino acid sequence numbering of the Pre-S1 region is based on subtype y composed of 108 amino acid residues, unless otherwise specified.

The L protein and the M protein have particle-forming abilities similar to the S protein. Therefore, the Pre-S1 and Pre-S2 regions may be optionally substituted, added, deleted and/or inserted. For example, a bio-nanocapsule that lacks a liver cell recognition ability can be produced by using a modified protein obtained by deleting a liver cell recognition site contained at positions 3 to 77 in the Pre-S1 region (subtype y). Further, the Pre-S2 region has a site capable of recognizing liver cells through albumin, and this albumin recognition site can also be deleted. In contrast, the S region (226 amino acid residues), which has a particle-forming ability, should be modified in such a manner that the particle-forming ability is not impaired. For example, when the positions 107 to 148 in the S region are deleted, the particle-forming ability is retained (J. Virol. 2002 76 (19), 10060-10063); therefore, this region may be substituted, added, deleted and/or inserted, etc. Similarly, when hydrophobic 154 to 226 residues at the C terminus are substituted, added, deleted and/or inserted, etc., the particle-forming ability can also be retained. In contrast, 8 to 26 residues (TM1) and 80 to 98 residues (TM2) in the S region are transmembrane helices (transmembrane sequences); therefore, it is desirable that this region not be mutated or that this region is deleted, added and/or substituted, etc. while maintaining the hydrophobic residues, so as to retain the transmembrane property.

In one preferable embodiment, modifications of the hepatitis B virus protein include any of the various modified proteins that have the ability to form bio-nanoparticles. Taking HBsAg protein as an example, any number of substitutions, deletions, additions and insertions may be included in the Pre-S1 and Pre-S2 regions. In the S region, one or more, for example, 1 to 120, preferably 1 to 50, more preferably 1 to 20, still more preferably 1 to 10, and particularly 1 to 5 amino acid residues may be substituted, added, deleted or inserted. Methods for introducing mutations such as substitution, addition, deletion and insertion, in DNA encording the above protein, include gene engineering techniques such as site specific mutagenesis (Methods in Enzymology, 154, 350, 367-382 (1987); ibid., 100, 468 (1983); Nucleic Acids Res., 12, 9441 (1984)), and chemical synthesis means such as the phosphate triester method and phosphate amidite method (for example, using a DNA synthesizer) (J. Am. Chem. Soc., 89, 4801 (1967); ibid., 91, 3350 (1969); Science, 150, 178 (1968); Tetrahedron Lett., 22, 1859 (1981)). The codon for use can be selected in consideration of the codon usage in the host.

In the case of hollow bio-nanoparticles composed of a liver cell-recognizing protein, such as L protein or M protein, as a hepatitis B virus protein or modified protein thereof, introducing a cell recognition site is not necessary. On the other hand, in the case of hollow bio-nanoparticles composed of a modified protein obtained by deleting the liver cell recognition site contained at 3 to 77 amino acid residues in the Pre-S1 region, or a modified protein obtained by deleting both Pre-S1 and Pre-S2 regions, the bio-nanoparticles per se cannot recognize cells. Therefore, a cell recognition site is introduced into the bio-nanoparticles so that cells other than liver cells can be recognized and nucleic acid can be introduced into various target cells. Cell recognition sites that recognize a particular cell are preferably, for example, cell function-regulating molecules composed of polypeptides such as growth factors and cytokines; cell surface antigens; tissue-specific antigens; polypeptide molecules such as receptors, which distinguish cells and tissues; polypeptide molecules derived from viruses and microorganisms; antibodies; and sugar chains. Specific examples thereof include antibodies against EGF receptors and IL-2 receptors that are specifically expressed in cancer cells; EGF; and receptors presented by HBV. Alternative examples include proteins (e.g., ZZ tag) capable of binding an antibody Fc domain, or StreptoTag, which exhibits a biotin-like activity to present a biorecognition molecule labeled with biotin via streptavidin.

The above ZZ tag is defined as an amino acid sequence having the ability to bind to the Fc region of immunoglobulin G, and is composed of the following two repetition sequence

(sequence of ZZ tag:
VDNKFNKEQQNAFYEILHLPNLNEEQRNAFIQSLKDDPSQSANLLAE
AKKLNDAQAPKVDNKFNKEQQNAFYEILHLPNLNEEQRNAFIQSLKD
DPSQSANLLAEAKKLNDAQAPK).

When the cell recognition site is a polypeptide, a hollow bio-nanoparticle that recognizes a target cell can be obtained by ligating a DNA encoding hepatitis B virus protein or a modified protein thereof to DNA encoding the cell recognition site in frame through DNA encoding a spacer peptide as needed, incorporating this into a vector or the like, and expressing it in an eukaryotic cell.

When the cell recognition site is an antibody, the desired hollow bio-nanoparticle can be obtained by ligating DNA encoding hepatitis B virus protein or a modified protein thereof to DNA encoding the ZZ tag in frame through DNA encoding a spacer peptide as needed, incorporating this into a vector or the like, expressing it in a eukaryotic cell, and mixing the resulting hollow bio-nanoparticle with an antibody capable of recognizing the target cell.

When the cell recognition site is a sugar chain, the desired hollow bio-nanoparticle can be obtained by adding (or attaching or linking) biotin to a hollow nanoparticle by chemical modification and presenting a sugar chain or the like labeled with biotin via streptavidin, etc.

The modification of the HBsAg protein may be modified proteins obtained by modifying antigenicity (modified proteins obtained by deleting/substituting a site involved in the antigenicity, such as an epitope), stability of the particle structure, cell selectivity, and the like. HBsAg (Q129R, G145R) particles described in Japanese Unexamined Patent Publication No. 2001-316298 have low antigenicity; therefore, these particles can particularly inhibit the production of antibodies against HBV and are preferably used to introduce materials, such as nucleic acids and drugs, into cells in the body.

The bio-nanocapsule comprising a modified HBsAg protein as mentioned above can be purified and preserved under the same conditions as used for bio-nanocapsules comprising an HBsAg protein such as L protein.

The method of disrupting eukaryotic cells may be any method that can disrupt the cells. Examples of such disruption methods include autolysis, enzymatic hydrolysis, acid digestion, ultrasonic disruption, homogenizers, high-pressure disruption, bead milling, grinding, freeze-thawing, and like methods. The bead milling and high-pressure disruption methods, and like methods are preferable.

The disrupted eukaryotic cell lysate is preferably subjected to heat treatment after removing water soluble materials and low-molecular-weight materials (e.g., saccharides, amino acids, oligopeptides, minerals) from the fraction containing bio-nanocapsules by dialysis.

The dialysis of the eukaryotic cell lysate can be carried out, for example, in the presence of a chelating agent such as 1 mM EDTA.

When disrupting the eukaryotic cells, it is preferable to inhibit eukaryotic cell-derived (e.g., yeast-derived) protease to prevent decomposition of the heat-resistant bio-nanocapsules. For this purpose, for example, urea is preferably used as a protease inhibitor. Urea is preferable to stabilize the molecules presented on the surface of the particles during the purification process. The protease inhibitor is preferably removed by a method such as dialysis before the heat treatment process to make an efficient heat treatment.

Subsequently, if necessary, the dialyzed lysate can be heat-treated to precipitate and remove the eukaryotic cell-derived contaminant proteins. The heat treatment temperature is about 65° C. to about 90° C., preferably about 65° C. to about 85° C., and more preferably about 70° C. to about 80° C.

When the heat treatment temperature is too low, it takes time to complete the treatment. When the temperature is too high, the antigen level is reduced, which may adversely affect the properties of the obtained bio-nanocapsules.

The heat treatment time is not particularly limited, as long as denaturation of the contaminant proteins occurs and the contaminant proteins are precipitated and removed. For example, the heat treatment time may be about 10 to about 60 minutes.

For example, Japanese Unexamined Patent Publication No. 252976/1993 describes purifying an HBsAg protein by heat treatment, in which the heat treatment temperature used is in the range of 45° C. to 60° C., and preferably 50° C. Japanese Unexamined Patent Publication No. 76370/1993 describes performing a heat treatment at 30° C. to 40° C., preferably at 37° C., for 1 to 6 hours to purify an HBsAg protein.

Generally, viruses are inactivated by heat treatment at 60° C. Additionally, in Chapter II, pages 371 to 398 of the 1992 WHO Global Blood Safety Initiative report “Virus Inactivation in Blood and Blood Products”, it is stated that a primary method for inactivating viruses is heating at 60° C. for 10 hours or more, and that most of the proteins are denatured by heat treatment in a liquid state at 60° C.

The heat treatment of the present invention is performed at 65° C. or more to produce bio-nanocapsules. If the heat treatment is performed at temperatures not higher than 60° C., the contaminated proteins cannot be sufficiently separated. Thus, such a low temperature is not preferable.

After the heat treatment, the denatured contaminant proteins are precipitated and separated by centrifugation, and bio-nanocapsules can be recovered as the supernatant. The centrifugation conditions may be, for example, centrifugation at a temperature of about 4° C. to about 8° C. at 34,780×g for 20 to 60 minutes.

Subsequently, the obtained supernatant-derived bio-nanocapsule particles are purified by chromatography. The chromatography methods are preferably affinity chromatography and/or gel filtration chromatography.

Preferable examples of affinity resins include resins having a sulfate group (sulfate esters), such as Sulfate-Cellulofine, Heparin-Cellulofine, and Heparin Sepharose CL6B (Pharmacia Corp.), hydroxyapatite resins, and anti-HBsAg antibody-bound resins, the anti-HBsAg antibody being produced by using HBsAg particles as an antigen. Affinity chromatography and gel filtration chromatography can be performed according to conventional methods.

The heat-resistant bio-nanocapsule of the present invention is preferably lyophilized to improve preservation stability. The lyophilization is preferably carried out, for example, after freezing, preferably quick-freezing, at about −20° C. or less. The quick-freezing is preferably carried out using a cooling medium such as liquid nitrogen, dry ice-alcohol (e.g., ethanol, methanol, propanol, etc.), or dry ice-acetone, which is capable of cooling to −70° C. or less, and more preferably −80° C. or less. Liquid nitrogen is particularly preferable.

Water can be used as a medium for the heat-resistant bio-nanocapsules before the freezing. Preferably, a buffer solution is used. Examples of buffer solutions include citrate buffer, phosphate buffer, succinate buffer, acetate buffer, and like buffer solutions. Phosphate buffer solutions are preferable. Such buffer solutions may contain salts such as NaCl.

The pH of the buffer solution is preferably in the range of about 6 to about 8, and more preferably about 6.5 to about 7.5.

An excipient (a stabilizing agent) is preferably used when lyophilizing the bio-nanocapsules. Examples of such stabilizing agents include monosaccharides, disaccharides, oligosaccharides, sugar alcohols, glycol, polyhydric alcohols, and the like. Specific examples thereof include glucose, mannose, galactose, sucrose, trehalose, glycerol, erythritol, arabitol, xylitol, sorbitol, mannitol, propylene glycol, polyethylene glycol, and the like. Monosaccharides such as glucose and disaccharides such as sucrose are particularly preferable.

The stabilizing agent is added to the bio-nanocapsule solution before lyophilization. The stabilizing agent may be added to a concentration of about 5 to about 40%, and preferably 5 to 20% to achieve better preservation stability.

The amount of stabilizing agent is about 1 to about 30 parts by weight, and preferably about 5 to about 20 parts by weight, per 100 parts by weight of the bio-nanocapsule particles.

Lyophilization is performed according to a conventional method. Lyophilization can be advantageously carried out, for example, under vacuum for one day and night, e.g., for 16 hours or more.

The lyophilized bio-nanocapsule is preferably stored at 10° C. or less, and more preferably at 4° C. or less.

### EXAMPLES

The present invention will be described in greater detail below using Examples; needless to say, however, the invention is not limited to these Examples.

The preparation method for each substance used in the Examples is described below.

### (i) 0.2 M PMSF Solution

| | |
|---|---|
| PMSF | 1.7149 g |

PMSF was diluted with an analytical-grade ethanol to make a total of 50 mL. The solution was stored at −20° C.

The solution was passed through a filter (0.22 μm) before use.

If precipitation was observed, the solution was completely dissolved in an ultrasonic cleaner before use.

### (ii) 10% Tween 80

| | |
|---|---|
| Tween 80 | 5.0 mL |

Tween 80 was diluted with MilliQ water to make a total of 50 mL. The solution was stored and used at 4° C.

The solution was passed through a filter (0.22 μm) before use.

(iii) Buffer A (w/o Tween 80)

| | | | | |
|---|---|---|---|---|
| Urea (for biochemistry, | 7.5 | M | 450.45 | g |
| Sigma 32-0291-8) | | | | |
| TRIZMA BASE | 0.1 | M | 12.11 | g |
| NaH₂PO₄—2H₂O | 50 | mM | 7.2 | g |
| EDTA-2Na | 15 | mM | 5.58 | g |

MilliQ water was added to the above components to make a total of 0.9 L, after which the mixture was adjusted to a pH of 7.4 with 1N HCl and then diluted to make a total of 1 L. The resulting buffer was stored at 4° C. and used at 4° C.

The buffer was passed through a filter (0.22 μm) before use.

PMSF (concentration when used: 2 mM) was also added as a protease inhibitor before use.

### (iv) PBS (×10)

| | | |
|---|---|---|
| NaCl | 80 | g |
| Na₂HPO₄—12H₂O | 28.9 | g |
| KCl | 2 | g |
| KH₂PO₄ | 2 | g |

The above components were diluted with MilliQ water to make a total of 1 L.

### (v) 0.2 M Na₂HPO₄

| | |
|---|---|
| Na₂HPO₄•12H₂O | 35.82 g |

The above component was diluted with MilliQ water to make a total of 0.5 L.

### (vi) 0.2 M NaH₂PO₄

| | |
|---|---|
| NaH₂PO₄•2H₂O | 15.6 g |

The above component was diluted with MilliQ water to make a total of 0.5 L.

(vii) 0.1 M Phosphate Buffer (pH 7.2)

| | |
|---|---|
| 0.2 M Na₂HPO₄ | 72 mL |
| 0.2 M NaH₂PO₄ | 28 mL |

The above components were diluted with MilliQ water to make a total of 200 mL.

(viii) Buffer A (+Tween 80), Prepared Before Use (Buffer for Cell Disruntinn)

| Buffer A (w/o Tween 80) | | 500 mL |
|---|---|---|
| 10% Tween 80 | 0.1% | 5 mL |

Before use, PMSF (concentration when used: 2 mM) and APMSF (concentration when used: 100 μg/mL) were added as protease inhibitors. The solution was passed through a filter (0.22 μm) before use.

### (ix) PBS, 1 mM EDTA (Buffer for Dialysis)

| | | | |
|---|---|---|---|
| PBS (x 10) | | 100 | mL |
| EDTA-2Na | 1 mM | 0.37 | g |

The above components were diluted with MilliQ water to make a total of 1 L.

The buffer was passed through a filter (0.22 μm) before use.

### (x) 10 mM Phosphate Buffer pH 7.2 (Buffer for Sulfate-Cellulofine)

| | | |
|---|---|---|
| 0.1 M phosphate buffer (PH 7.2) | 10 mM | 100 mL |

The above component was diluted with MilliQ water to make a total of 1 L.

The solution was passed through a filter (0.22 μm) to remove air before use.

### (xi) 10 mM Phosphate Buffer pH 7.2+2 M NaCl (Elution Buffer for Sulfate-Cellulofine)

| | | | | |
|---|---|---|---|---|
| 0.1 M Phosphate Buffer (PH 7.2) | 10 | mM | 100 | mL |
| NaCl | 2 | M | 116.88 | g |

The above components were diluted with MilliQ water to make a total of 1 L.

The buffer was passed through a filter (0.22 μm) to remove air before use.

(xii) PBS, 1 mM EDTA (Buffer for Gel Filtration)

| | | | |
|---|---|---|---|
| PBS (x 10) | | 100 | mL |
| EDTA-2Na | 1 mM | 0.37 | g |

The above components were diluted with MilliQ water to make a total of 1 L.

The buffer was passed through a filter (0.22 μm) to remove air before use.

### Example 1

HBsAg particles (bio-nanocapsules) were purified in accordance with the procedures described below.

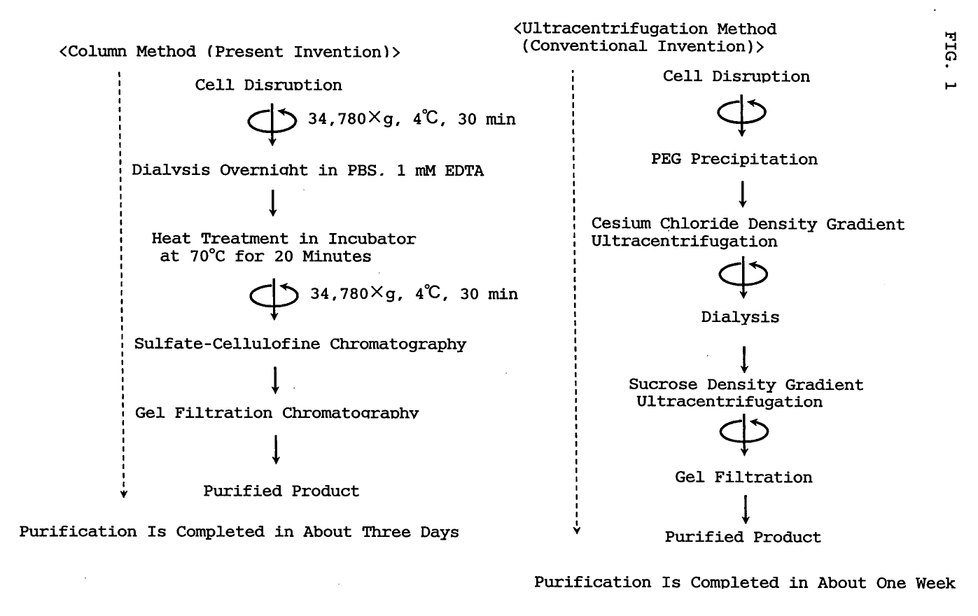

FIG. 1 shows the outline of the purification method.

### (1) Cell Disruption

HBsAg particles to be disrupted were L protein particles.

The container, as well as the buffer and beads to be added were cooled to 4° C. before use. 15 to 25 g of cells (corresponding to about 1 L of culture) stored at −80° C. were suspended in 160 mL of Buffer A (+Tween 80+PMSF 4 mM+APMSF 100 μg/mL) using a pipette. The suspension was added together with 175 mL of glass beads (diameter: 0.5 mm) to a container for a BEAD-BEATER (volume: 350 mL, BioSpec Products, Inc.), after which the suspension was disrupted in ice for 2 minutes, cooled for 1 minute, disrupted for 2 minutes, cooled for 1 minute, and disrupted for 1 minute, and then cooled until the heat was removed from the container. After collection of the supernatant, the beads were washed with a small amount (about 40 mL) of Buffer A to collect the lysate, and this procedure was repeated twice. The collected lysate was centrifuged (34,780×g, 30 min, 4° C.), and then the supernatant was collected.

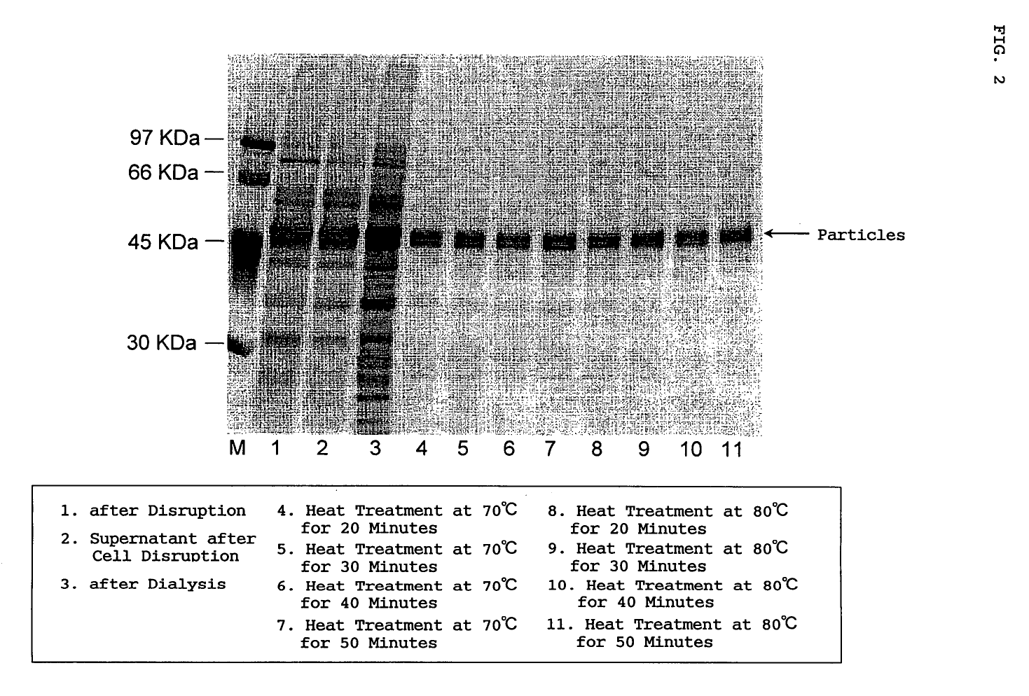

FIG. 2 shows the results of SDS-PAGE performed after cell disruption and on the supernatant.

### (2) Dialysis

The sample was dialyzed against about a 20-fold volume of the external solution (PBS, 1 mM EDTA) in a low-temperature room for 1 hour, after which the external solution was replaced with an equal amount of the external solution, and then dialysis was continued for 1 hour. This procedure was repeated a total of four times (a total of 4 hours), and then dialysis was continued for 2 hours after the external solution was replaced with about a 20-fold volume of the external solution as above.

FIG. 2 shows the result of SDS-PAGE after dialysis.

### (3) Heat Treatment

The dialyzed sample was dispensed in about 40 mL portions into centrifugation tubes, and heat-treated in an incubator preheated to 70° C. for 20 minutes. After heat treatment, the samples were centrifuged (34,780×g, 30 min, 4° C.) and the supernatants were collected.

Similarly, each sample was heat-treated at 70° C. for 30, 40, or 50 minutes, or heat-treated at 80° C. for 20, 30, 40, or 50 minutes, and measured for the amount of protein (mg) and amount of antigen (unit) before and after the heat treatment. The results are shown in Table 1. FIG. 2 shows the results of SDS-PAGE after heat treatment under these conditions.

TABLE 1

| | | Amount of Protein (mg) | | Amount of Antigen (Unit) | | |
|---|---|---|---|---|---|---|
| | | A: Before Heat | B: After Heat | C: Before Heat | D: After Heat | D/C |
| ° C. | min. | Treatment | Treatment | Treatment | Treatment | B/A |
| 70 | 20 | 50.3 | 13.4 | 9120 | 8265.6 | 3.4 |
| | 30 | 50.3 | 11.6 | 9120 | 9688 | 4.6 |
| | 40 | 50.3 | 12.7 | 9120 | 7789.6 | 3.4 |
| | 50 | 50.3 | 13.0 | 9120 | 9900.8 | 4.2 |
| 80 | 20 | 50.3 | 13.4 | 9120 | 8136.8 | 3.3 |
| | 30 | 50.3 | 8.9 | 9120 | 7263.2 | 4.5 |
| | 40 | 50.3 | 13.1 | 9120 | 8164.8 | 3.4 |
| | 50 | 50.3 | 11.7 | 9120 | 6720 | 3.2 |

### (4) Sulfate-Cellulofine Chromatography

An AKTA System (Amersham Biosciences Corp.) was used.

The heat-treated samples were passed through a 0.45 μm filter, and then added to a cellulofine sulfate column (column diameter: 1.6×20 cm; medium: CHISSO CORP.; flow rate: 4 mL/min) equilibrated with 10 mM phosphate buffer pH 7.2+0.15 M NaCl. Unadsorbed fractions were washed with a volume of buffer double the volume of the column, eluted stepwise with 10 mM phosphate buffer pH 7.2+1 M NaCl, and fractionated into 4 mL portions. The eluate was measured for absorption at 280 nm and 260 nm. The amount of HBs antigen in each fraction was examined with IMx (ABBOTT JAPAN CO., LTD), and fractions containing HBs antigen were collected.

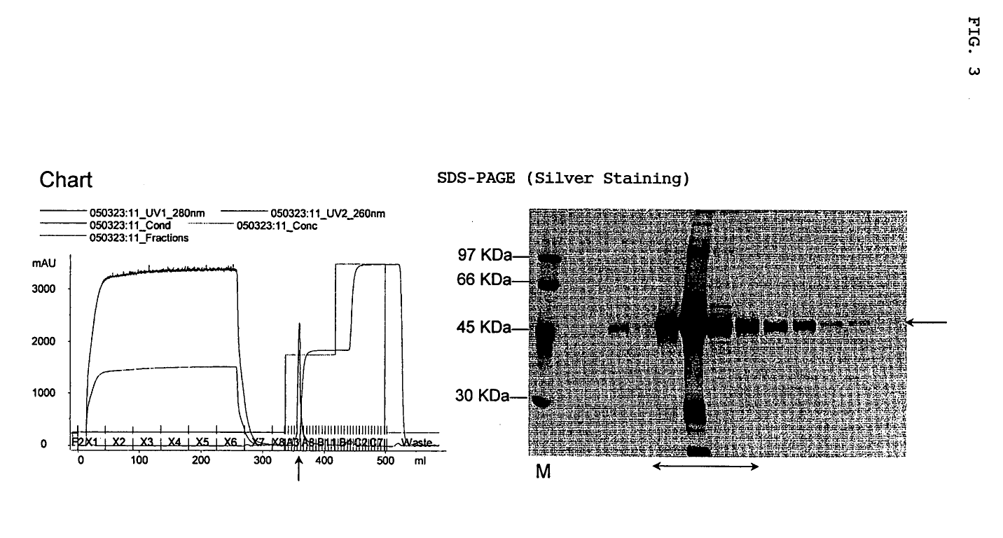

FIG. 3 shows the results of Sulfate-Cellulofine chromatography. The fraction indicated by the arrow shown in the chart of FIG. 3 was collected and passed through a gel filtration column.

The chromatography conditions were as follows.
Column: Sulfate-Cellulofine (1.6×20 cm)
Buffer: 10 mM Na phosphate (pH 7.2)
Elution buffer: 10 mM Na phosphate (pH 7.2), 2 M NaCl
Flow rate: 4 mL/min
Gradient: 0.15→0.5 M NaCl (step)
0.5→2 M NaCl (step)
Fraction: 5 mL/tube

### (5) Ultrafiltration

The sample after Sulfate-Cellulofine chromatography was concentrated (Amicon Ultra; NMWL 100K, MILLIPORE) to 5% or less of the volume of the gel filtration column.

### (6) Gel Filtration Chromatography

An AKTA System (Amersham Biosciences Corp.) was used.

The concentrated samples were passed through a 0.45 μm filter, added to a gel filtration column (column diameter: 1.6×60 cm; medium: Sephacryl S-500-HR; Amersham Biosciences Corp.; flow rate: 0.5 mL/min) equilibrated with PBS and 1 mM EDTA, eluted with the same buffer, and fractionated into 5 mL portions. The eluate was measured for absorption at 280 nm and 260 nm. The amount of HBs antigen in each fraction was examined with IMx (ABBOTT JAPAN CO., LTD), and also considering the results of SDS-PAGE, fractions containing HBs antigen were collected.

The chromatography conditions were as shown below.
Column: Sephacryl S-500 HR (1.6×70 cm)
Buffer: PBS, 1 mM EDTA
Flow rate: 0.5 mL/min
Fraction: 5 mL/tube

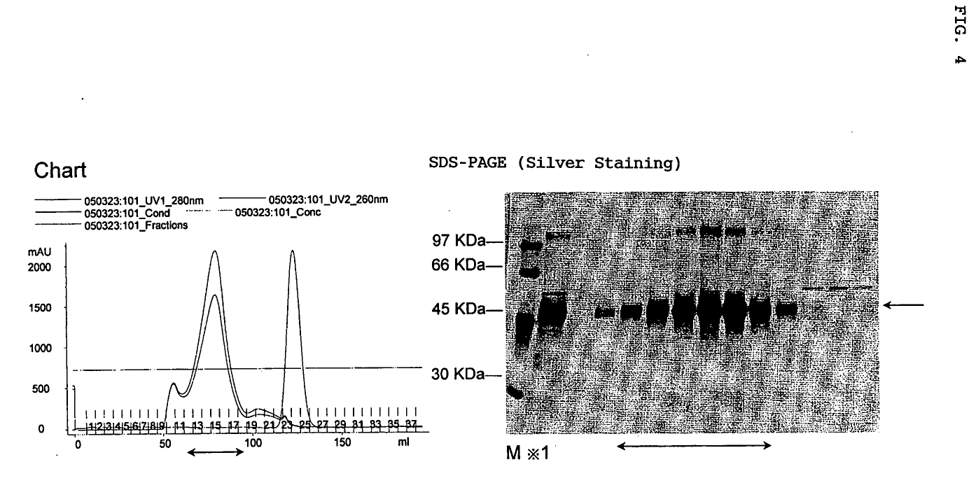

FIG. 4 shows the results of gel filtration chromatography.

### (7) Ultrafiltration and Filter Sterilization

The samples obtained after gel filtration were concentrated to a protein concentration of 0.2 mg/mL or more (Amicon Ultra; NMWL 100K, MILLIPORE). The concentrated samples of the fractions indicated by the arrow shown in the chart of FIG. 4 were collected and sterilized with a 0.45 μm filter for use as purified samples.

### (8) Results

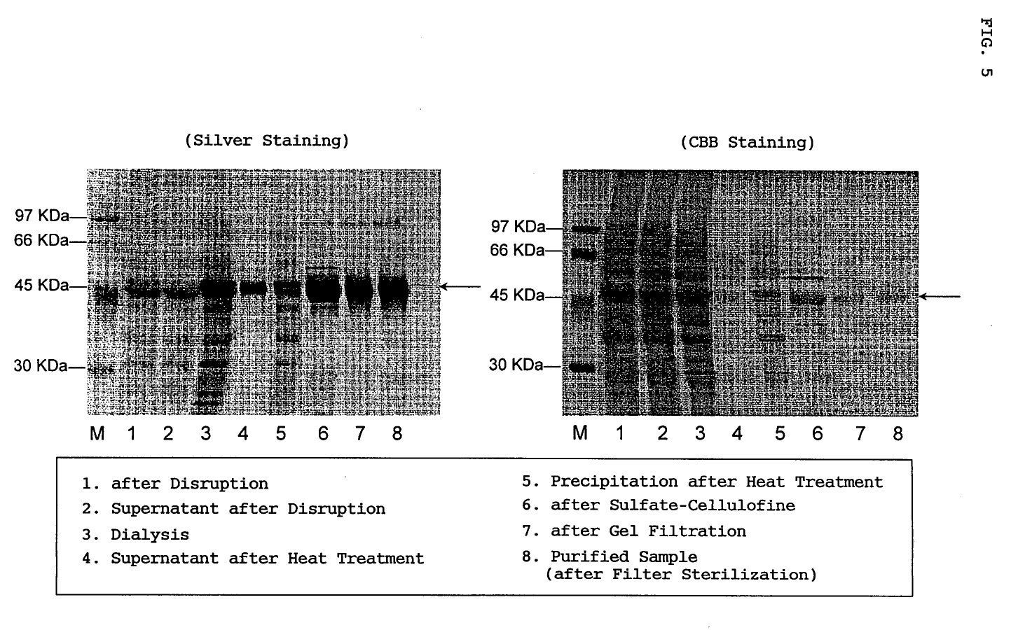

Table 2 shows the results of purification, and FIG. 5 shows the results of SDS-PAGE throughout all the steps. As shown in FIG. 5, two types of staining methods were employed: silver staining makes it easier to visualize particles, and CBB staining makes it easier to visualize contaminating proteins.

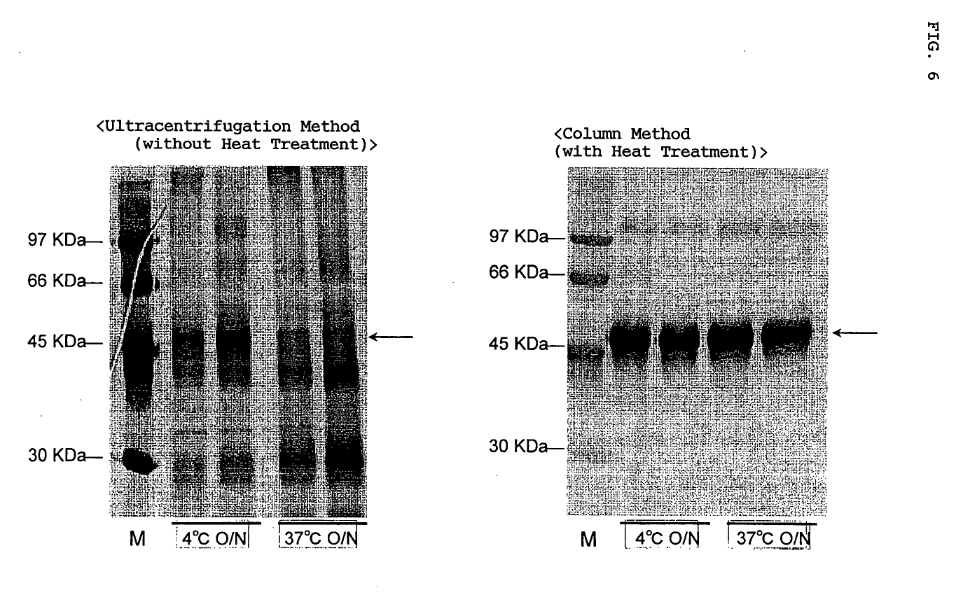

In addition, in order to evaluate the preservation stability of the particles, the fractions obtained after gel filtration were allowed to stand overnight at 4° C. or 37° C., and the decomposition thereof by protease was examined. The results are shown in FIG. 6. The results in FIG. 6 show that decomposition proceeded in the samples not subjected to heat treatment, whereas the heat-treated samples were not decomposed, revealing that the purification method of the invention is effective in obtaining nanocapsules resistant to decomposition by protease.

Table 3 shows comparisons of the average values for the method of the invention and a conventional method.

TABLE 2

| | Amount of | *1 Total | *2 | Specific |
|---|---|---|---|---|
| | Protein | Amount of | Recovery | Activity |
| Step | (mg) | Antigen (Unit) | (%) | (Unit/mg) |
| After | 2775.4 | 170931.6 | 100 | 61.6 |
| Disruption | | | | |
| Supernatant | 1488.3 | 154895.8 | 91 | 104.1 |
| After | | | | |
| Disruption | | | | |
| After | 1200.1 | *3 174272.0 | *3 102 | 145.2 |
| Dialysis | | | | |
| Supernatant | 139.7 | 106529.2 | 62 | 762.6 |
| After Heat | | | | |
| Treatment | | | | |
| After | 17.6 | 27108.0 | 16 | 1543.6 |
| Cellulofine | | | | |
| Sulfate | | | | |
| After Gel | 8.1 | 27204.0 | 16 | 3361.0 |
| Filtration | | | | |
| (Purified | | | | |
| Samples) | | | | |

*1 Equation for calculating the total amount of antigen Total amount of antigen (Unit) = (amount of samples: mL) × (IMx RATE value) × (dilution factor of IMx)/1000
*2 Equation for calculating the recovery Recovery (%) = (total amount of antigen in each step)/(total amount of antigen after disruption) × 100
*3 The value after dialysis is higher than the value for the supernatant after disruption because the amount of sample increased during dialysis, and is therefore within the margins of error.

TABLE 3

| | Ultracentrifugation | Column |
|---|---|---|
| | Method | Method |
| Recovery (%) | 1.8 | 15.5 |
| Specific Activity | 1971 | 3930 |
| (Unit/mg) | | |

### Example 2

Purified bio-nanocapsules (concentration: 500 μg/mL) were dispensed into 1.5 mL tubes in 400 μl portions, and glucose was added thereto to give a final concentration of 5 wt %. The 1.5 mL tubes were then immersed in liquid nitrogen prepared beforehand and rapidly frozen. After confirming that the bio-nanocapsules in the tubes were sufficiently frozen, they were dried in a vacuum dryer for a day. The dried samples were stored at 4° C.

Moreover, sucrose and trehalose were each added to the solution instead of glucose to give a final concentration of 10 wt %, and these bio-nanocapsules were similarly lyophilized.

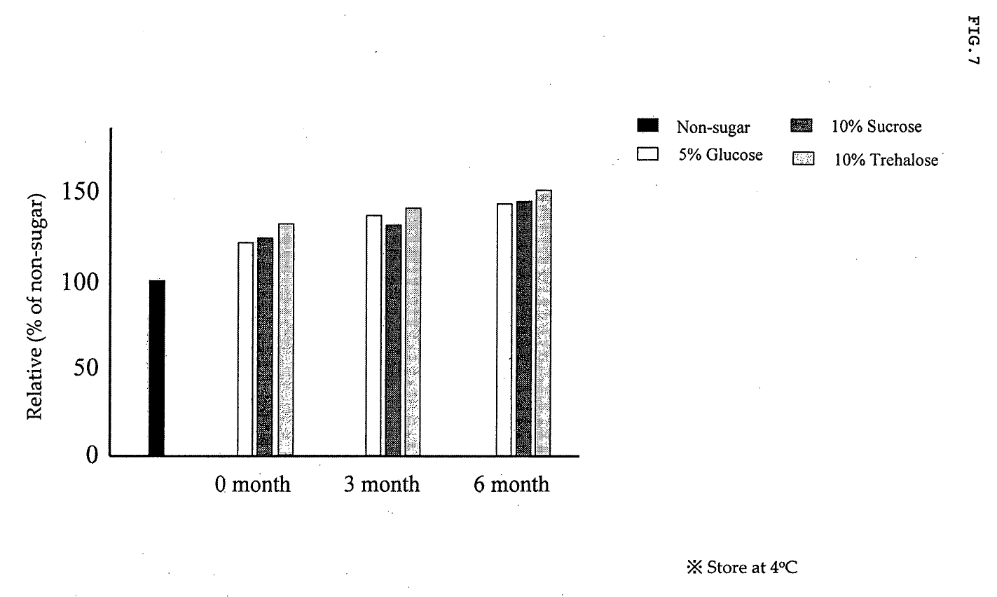

The lyophilized products and the control lyophilized products containing no saccharide were lyophilized at 4° C. The results are shown in FIG. 7.

The results of FIG. 7 clarify that the addition of saccharides enhances the preservation stability of the lyophilized products.

1-10. (canceled)

11. A method for purifying a capsule composed mainly of a hepatitis B virus surface antigen (HBsAg) protein produced by using a eukaryotic cell, the method comprising heat-treating a eukaryotic cell lysate at 65° C. to 90° C. to remove contaminant proteins, and then subjecting the resulting product to chromatography.

12. A method according to claim 11, wherein the chromatography comprises affinity chromatography and/or gel filtration chromatography.

13. A method according to claim 12, wherein the affinity chromatography is sulfate-cellulofine chromatography using a sulfate group-containing medium.

14. A method according to claim 12, wherein the affinity chromatography is antibody affinity chromatography using a medium having an anti-HBsAg antibody bound thereto, the anti-HBsAg antibody being produced by using HBsAg particles as an antigen.

15. A method according to claim 12, wherein the affinity chromatography is chromatography using hydroxyapatite as a medium.

16. A method for preserving a capsule composed mainly of a hepatitis B virus surface antigen (HBsAg) protein, comprising of freezing the capsule obtained by the method according to claim 11, followed by drying under vacuum.

17. A method according to claim 16, wherein the capsule is quickly frozen with a stabilizing agent.

18. A method according to claim 16, wherein the quick-freezing temperature is not higher than −80° C.
